(19) United States
(12) Patent Application Publication
(10) Pub. No.: US 2024/0065338 A1
(43) Pub. Date: Feb. 29, 2024

(54) USER INTERFACE FOR CONSUMABLE MEDIA DOSING APPARATUS

(71) Applicant: Wellness Insight Technologies, Inc., San Jose, CA (US)

(72) Inventor: Ramon Alarcon, Los Gatos, CA (US)

(21) Appl. No.: 18/238,417

(22) Filed: Aug. 25, 2023

Related U.S. Application Data
(60) Provisional application No. 63/401,051, filed on Aug. 25, 2022.

(51) Int. Cl. A24F 40/60; A24F 40/42; A24F 40/50; A24F 40/65

(52) U.S. Cl. CPC A24F 40/60; A24F 40/42; A24F 40/50; A24F 40/65; A61M 11/007

(57) ABSTRACT

A media dispensing device, comprises a vaporizer drive circuit; one or more memories; a touch display screen; and a control circuit comprising a processor coupled with the one or more memories configured to run the instructions, the instructions configured to cause the processor to: receive a dose target for a constituent via the touch display screen; determine whether to perform compensation for an inhalation media dose in order to ensure the dose target is met; when compensation is to be performed, determine the properly compensated inhalation media dose based on an associated compensation value for the constituent; and control the vaporizer drive circuit so as to dispense a compensated dose of the inhalation media.

### RELATED APPLICATION INFORMATION

This application claims priority under 35 U.S.C. § 119(e) to U.S. Provisional Application No. 63/401,051, filed Aug. 25, 2022, which is incorporated herein by reference in the entirety as if set forth in full. This Application is also related to U.S. patent application Ser. No. 16/520,148, filed Jul. 23, 2019, entitled “Systems for Analyzing and Controlling Consumable Media Dosing Information,” and U.S. patent application Ser. No. 18/211,072, filed Jun. 16, 2023, entitled “Systems for Analyzing and Controlling Consumable Media Dosing Information,” both of which are incorporated herein by reference in the entirety as if set forth in full.

### FIELD OF THE DISCLOSURE

The present disclosure relates to a method and apparatus for delivering doses of an inhalation media, and more particularly for a graphical user interface for an inhalation media device.

### BACKGROUND OF THE DISCLOSURE

Personal media dispensing devices, such as vaporizers are a popular alternative to traditional cannabis and tobacco leaf-based cigarettes that must be combusted in order to generate smoke for inhalation. Personal media dispensing devices can provide a vapor for inhalation, but do not produce certain byproducts of combustion that can be harmful to human health, or can be used to precisely control the dosing of a variety of media such as medicines, drugs, or supplements. Personal media dispensing devices can be electronic inhalers that vaporize or atomize a liquid solution into an aerosol that can then be delivered to a user. Such a typical media dispensing devices can have two main parts—1) a housing containing a battery and control electronics and 2) a liquid storage component. The housing, which can hold a battery, such as a rechargeable lithium-ion (Li-ion) battery and an activation sensor. The liquid storage component typically includes a liquid solution, an atomizer and a mouthpiece, although the atomizer can reside in the housing in certain configurations. The atomizer typically includes a heating element that vaporizes the liquid solution. Certain advanced media dispensing devices can also have the ability to communicate with a computer network, typically via a wireless connection to a mobile phone.

### SUMMARY OF THE DISCLOSURE

According to an aspect of the disclosure, a media dispensing device, comprises a drive circuit; a touch display screen; one or more memories configured to store a percentage of at least one constituent in an inhalation media, one or more compensation values for at least one compensation category for the at least one constituent, and instructions; and a control circuit comprising a processor coupled with the one or more memories configured to run the instructions, the instructions configured to cause the processor to: receive a dose target for a constituent via the touch display screen; determine whether to perform compensation for an inhalation media dose in order to ensure the dose target is met; when compensation is to be performed, determine the properly compensated inhalation media dose based on an associated compensation value for the constituent; and control the drive circuit so as to dispense a compensated dose of the inhalation media.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1A shows an example of a media dispensing device according to an embodiment of the disclosure;

FIGS. 1B-1D show a main menu of a user interface of a media dispensing device application, according to an embodiment of the disclosure;

FIGS. 2A-2P show media dispensing device application screen related to dose selection, according to an embodiment of the disclosure;

FIG. 3 shows a device control scheme which may be implemented by the media dispensing device application, according to an embodiment of the disclosure;

FIGS. 4A-4D show media dispensing device application screens related to product information, according to an embodiment of the disclosure;

FIG. 5A-5E show media dispensing device application and mobile device application screens, according to an embodiment of the disclosure;

FIGS. 6A-6C show media dispensing device application and mobile device application screens, according to an embodiment of the disclosure;

FIG. 7A-7B show media dispensing device application screens, according to an embodiment of the disclosure;

FIG. 8A-8B show media dispensing device application and mobile device application screens, according to an embodiment of the disclosure;

FIG. 9 illustrates an example infrastructure, in which one or more of the processes described herein, may be implemented, according to an embodiment;

FIG. 10 illustrates an example processing system, by which one or more of the processes described herein, may be executed, according to an embodiment;

FIGS. 11A-k illustrate screen shots presented by an application that can run a user system/device in the architecture of FIG., as opposed to the device of FIG. 1B-D, related to functionality provided by such an application; and

FIGS. 12A-C illustrate user interface screens that can be presented to, e.g., a coach to allow the coach to monitor and direct users, and a distributor to allow them to ensure media availability for users within the architecture of FIG. 9.

### DETAILED DESCRIPTION OF THE DISCLOSURE

The disclosure and the various features and advantageous details thereof are explained more fully with reference to the non-limiting embodiments and examples that are described and/or illustrated in the accompanying drawings and detailed in the following. It should be noted that the features illustrated in the drawings are not necessarily drawn to scale, and features of one embodiment can be employed with other embodiments as the skilled artisan would recognize, even if not explicitly stated herein. Descriptions of well-known components and processing techniques can be omitted so as to not unnecessarily obscure the embodiments of the disclosure. The examples used herein are intended merely to facilitate an understanding of ways in which the disclosure can be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

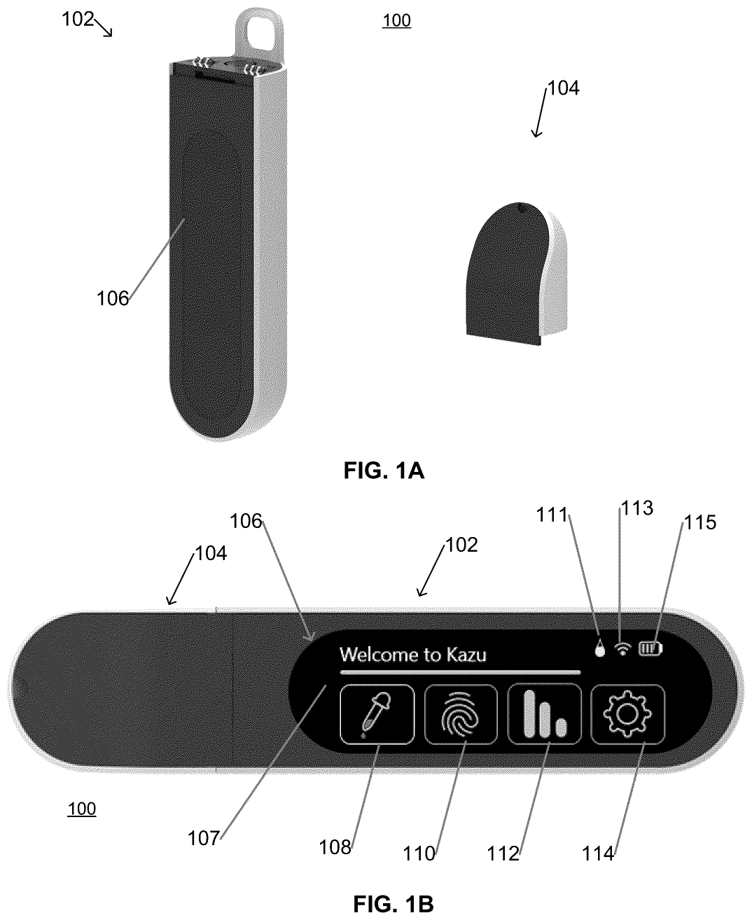

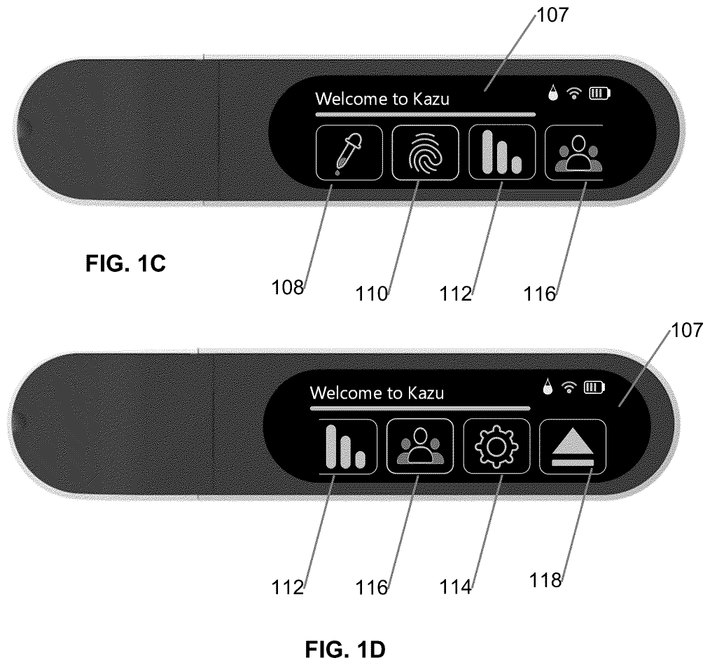

FIG. 1A shows an example of a media dispensing device 100 according to an aspect of the disclosure. In the instant example, the media dispensing device 100 is comprised of a body/housing 102 (including dose/vaporizer controller) and a media containing cartridge/pod 104.

In an exemplary embodiment, the body 102 of the media dispensing device includes a touch panel display screen 106. The display screen 106 can be a capacitive or pressure sensing surface capable of detecting the position of the user's finger and/or gestures created by the motion of the user's finger and interpreting such input for the purpose of navigating and/or selecting items on the display screen 106.

The display screen 106 can display information and provide for device controls including, but not limited to: the amount of inhalation media remaining in the cartridge 104, the charge status of the battery, the status/strength of wireless communication, the amount of inhalation media to be dosed, characteristic information about the inhalation media, operational status of the vaporizer controller, response to dosing commands, options for dosing, dosing inputs, health statistics, advertisements, brand logos, command input options, screen brightness controls, charging options, vaporizer settings, maintenance functions, message notifications, messages, dose sharing options, social media messages, and power options. In an exemplary embodiment, touch display screen 106 can be used to navigate through and/or select information/items displayed directly on the screen 106, and provide input to the device 100.

In certain embodiments, the touch screen 106 can be equipped with or used as a fingerprint reading sensor that can, for example, unlock/enable the device 100, according to a list of authorized users and associated fingerprints stored in memory. Alternatively, or additionally, the user can also unlock/enable the device 100 by entering/drawing a security pattern associated with an authorized user on the display screen 106. In some embodiments, some of the screens/information may be accessible without authorization (e.g., fingerprint reading or security pattern) while other screens/information are accessed only with authorization.

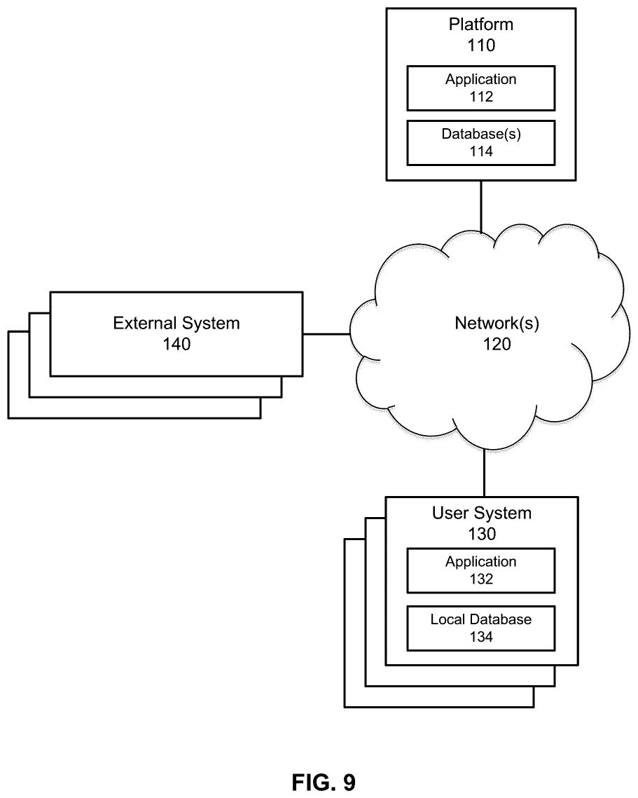

In an exemplary embodiment, the media dispensing device 100 includes or is in communication with one or more client applications 132 (see FIG. 9) that have a user interface for enabling the user to interact with the media dispensing device via the display 106. In some embodiments, some or all functions of the application(s) may be included on one or more user computer devices (e.g., personal computer, mobile phone, etc.), such that a user interface on a user computer device mirrors or supplements the user interface provided on the media dispensing device 100.

In an exemplary embodiment, the media dispensing device 100 has the ability to send information to a network 120 and/or another user device. Appropriate communication methods include, but are not limited to: USB, Bluetooth, Zigbee, Wi-Fi, and digital cellular.

FIGS. 1B-1D show the user interface of the application 132 showing a graphical main menu 107 of the application 132, including a plurality of main menu options. In an exemplary embodiment, FIG. 1B shows the main menu having options including: (i) dosing mode 108, (ii) current inhalation media information 110, (iii) personal history/progress 112, and (iv) settings 114. The main menu options may be represented with appropriate icons/touch screen buttons. Alternatively or additionally, FIGS. 1C-1D show a main menu of the application 132 user interface having options including: social/friends aspects 116 and additional options 118. In one embodiment, one or more options of the main menu may be accessible via scrolling/swiping (e.g., right or left) as shown in FIGS. 1C-1D.

The display 106 may include additional information, for example, current pod status 111, local network status 113 (e.g., Wi-Fi connection strength), and current battery status 115.

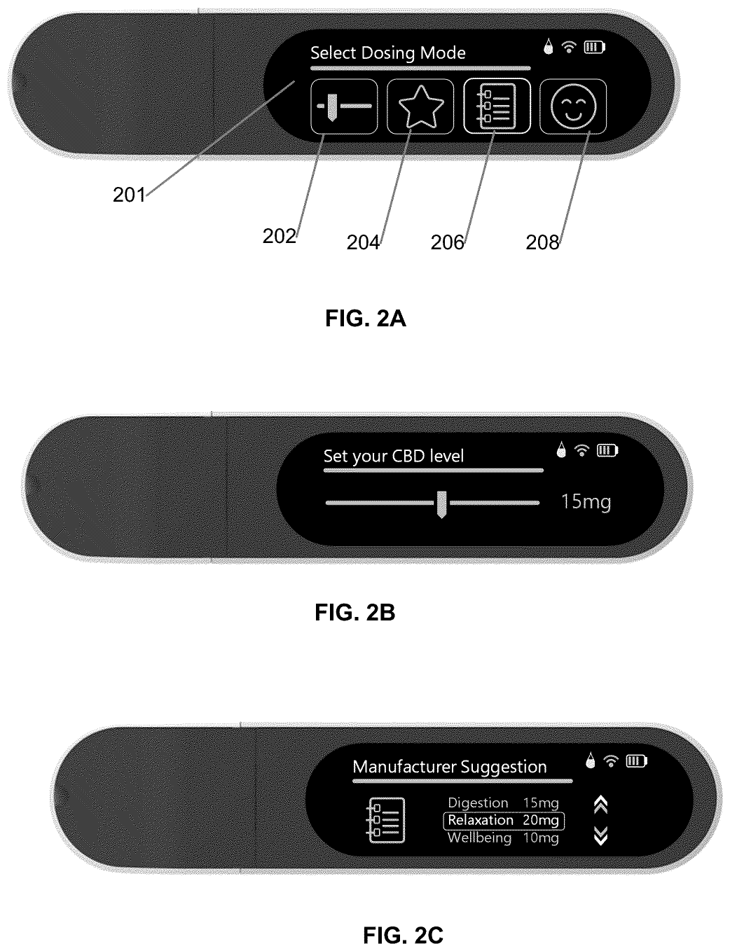

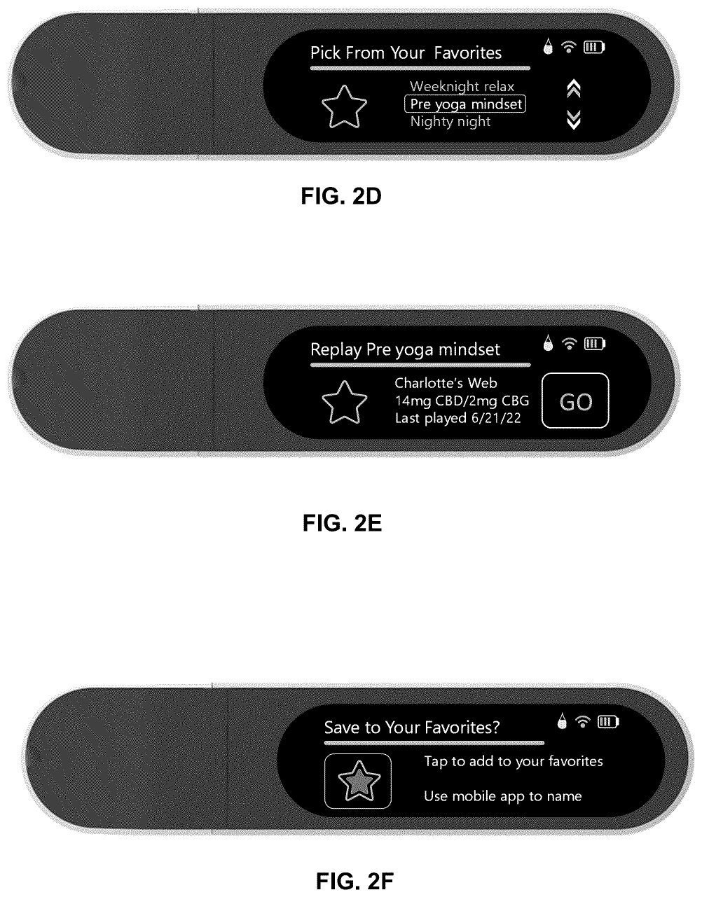

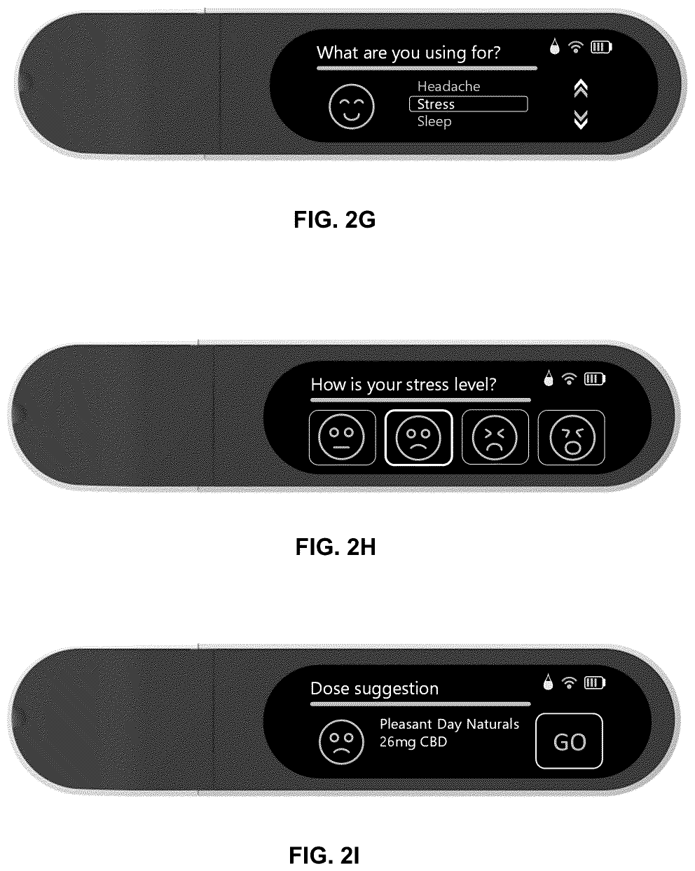

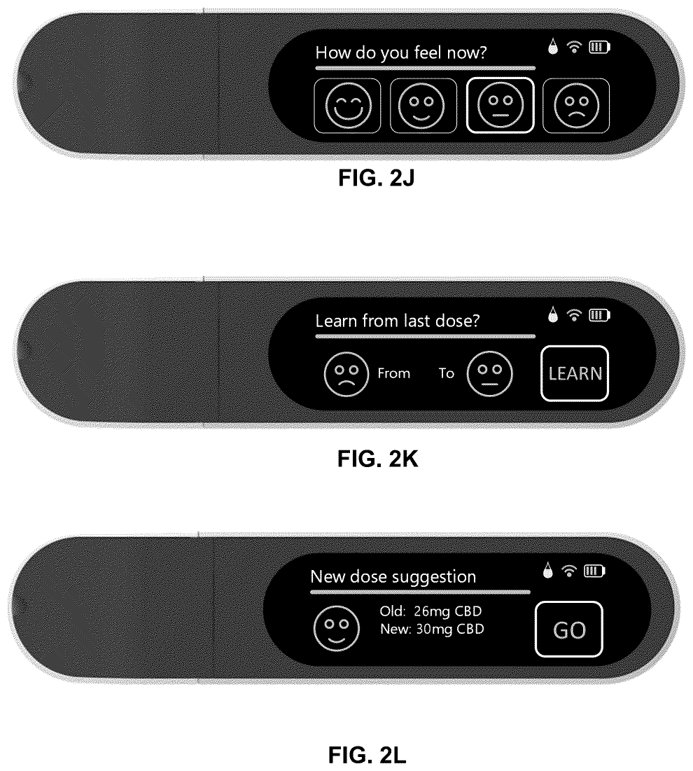

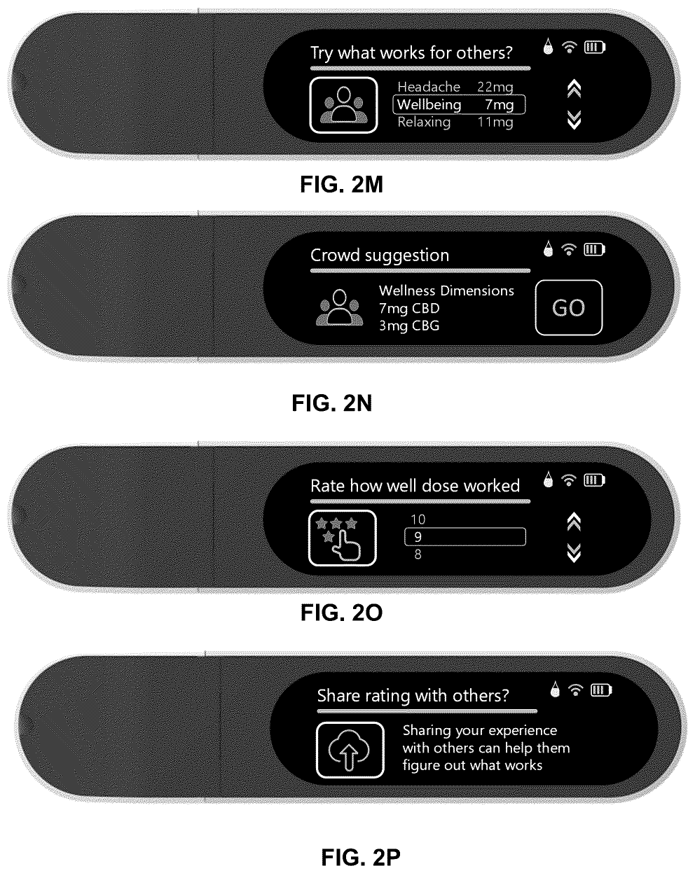

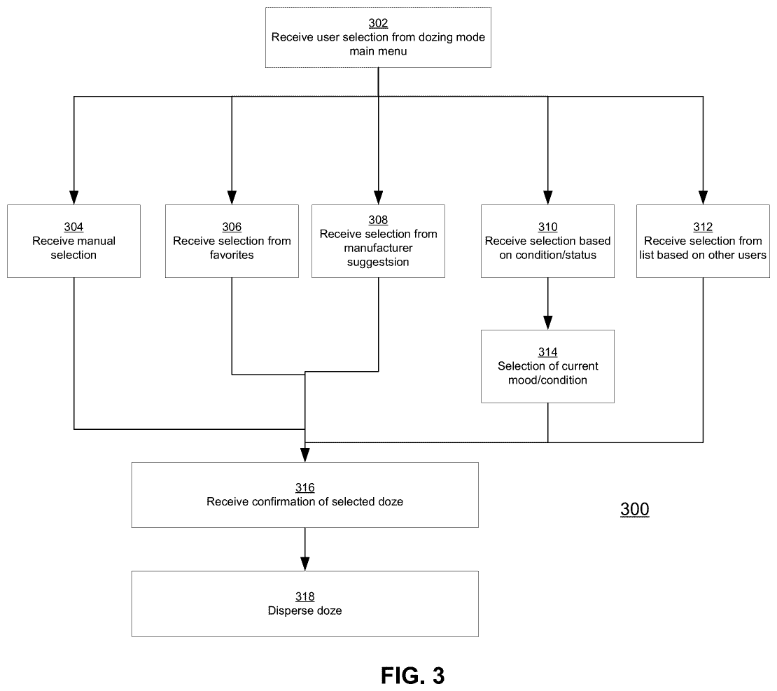

FIGS. 2A-2P show the user interface of the application 132 corresponding to the dosing mode 108, including functionality related to: selecting a dose of inhalation media, dispersing the dose, and providing feedback related to a dispersed dose. FIG. 3 shows a flow diagram of a scheme showing a process of receiving a user dose selection via the application 132. Again, while the media disclosed in the examples of FIGS. 2A-2P illustrate CBD as the media, the media can be medications, drugs, supplements, etc.

FIG. 2A shows an example of a dosing mode menu 201 of the application 132 which may be accessed, for example, by selecting dosing mode option 108 from the main menu 107. In an exemplary embodiment, the dosing mode menu 201 includes options (e.g., represented by icons/touch screen buttons) for: (i) setting inhalation media (e.g., CBD) level manually 202, (ii) accessing previously saved dose settings (e.g., favorites) 204, (iii) accessing manufacturer dose suggestions/recommendations, and (iv) accessing dose recommendations according to a user's current or intended condition/status 208.

FIG. 2B shows an example of a screen allowing a user to manually set an amount/dose of inhalation media (e.g., a “Set your CBD level” screen). In an exemplary embodiment, the screen shows a sliding control that allows users to select a specific value of CBD, however, other UI controls for selecting a value may be used (e.g., a number keypad, a list of options, etc.).

FIG. 2C shows an example of a screen with manufacturer suggested dose options for various purposes.

FIG. 2D shows a list of previously saved/favorite doses, with more optionally available via scrolling. FIG. 2E confirmation window for a specific dose (“Replay Pre yoga mindset”) selected from favorites by a user. In an exemplary embodiment, the confirmation window includes the name given to the dose, the brand/label of the particular inhalation media (“Charlotte's Web”), the specific amounts of chemicals (e.g., CBD, CBG) in the dose, and the previous date of the specific dose. The confirmation window enables a user to cause the vaporizer device 10 to deliver the precise dose of inhalation media to the user (e.g., by pressing a “GO” button on the touch screen).

FIG. 2F shows an example of a screen suggesting/asking whether the previous dose should be added to favorites. The screen may appear to the user after a dose has been distributed.

FIGS. 2G-2H show dose recommendations according to a user's current or intended condition. Thus, for example, if the user selects in the screen shot of FIG. 2G that they are using the device 100, and the media dispensed thereby, for stress, then as illustrated the screen shot of FIG. 2H, the device 100 can be configured to illicit how the user is feeling, with respect to stress at the moment. As illustrated in FIG. 2I, device 100 can then be configured to suggest a dose based on the user feedback/input.

FIGS. 2J-2L show examples of after dose user prompts that may be presented a predetermined time period after a dose (e.g., an hour after the dose has been distributed). In one embodiment, the application may prompt a user for their current mood/feeling/condition. FIG. 2J shows an example of a multiple choice mood/feeling prompt in picture form, however, the prompt choices may additionally or alternatively be text or audio based. In some embodiments, the application 132 can learn, for example, adjust its recommendations by learning from the user's experience (FIGS. 2K-2L), which can then influence the suggestion presented in step 310 and illustrated in FIG. 2I.

In some embodiments, the application 132 may be used to connect other users of similar devices/applications (e.g., a user's social network, all or a portion of all users of the application 132, etc.). FIG. 2M shows a list of dose suggestions obtained from others for various purposes (e.g., headache, wellbeing, relaxing, etc.). FIG. 2N shows confirmation window for a specific dose selected from the list in FIG. 2M. After a dose has been dispersed (e.g., a predetermined time period after), the user may be provided with a feedback window (FIG. 2O) and an option to share the feedback with others.

FIG. 3 shows a device control scheme 300 that describes how a media dispensing device 100 may be controlled to disperse a dose of inhalation media, starting from the main dozing mode menu. The control scheme 300 can be executed by a program (e.g., application 132) or plurality of functions programmed to be executed by one or more hardware processors of the media dispensing device 100. At block 302, a user may select an option from a “Select Dozing Mode” menu of the application (FIG. 2A). After receiving the user selection in block 302, the application moves onto to display the selected dose mode menu/screen. Examples of different dose mode screens are shown in FIG. 2B (manual selection), 2C (manufacturer suggested), 2D (favorites), 2G (condition/use), and 2M (social/crowd suggestions). The user selects a particular dose via one of the dose selection paths 304, 306, 308, 310/314, and 312, which leads to a confirmation window for a specific/selected dose (e.g., FIG. 2E, 2I, 2N). At block 316, the application receives confirmation for the selected dose and, at block 218, the media dispensing device 100 disperses the selected dose.

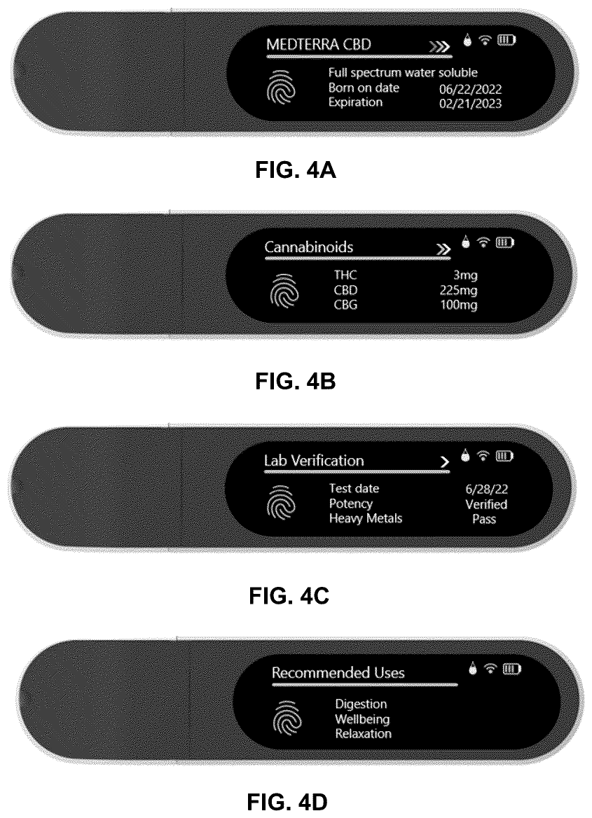

FIGS. 4A-4D show screen examples of the current product/inhalation media information that may be provided by the manufacturer/supplier. In an exemplary embodiment, the product information can be accessed via a button/icon 110 on a main menu (FIGS. 1B-1D) of the display 106.

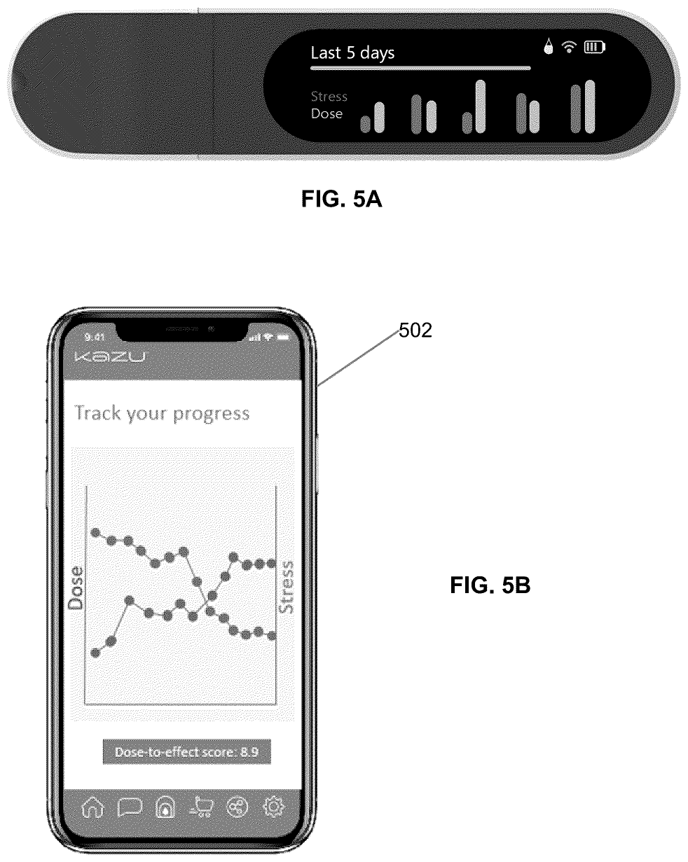

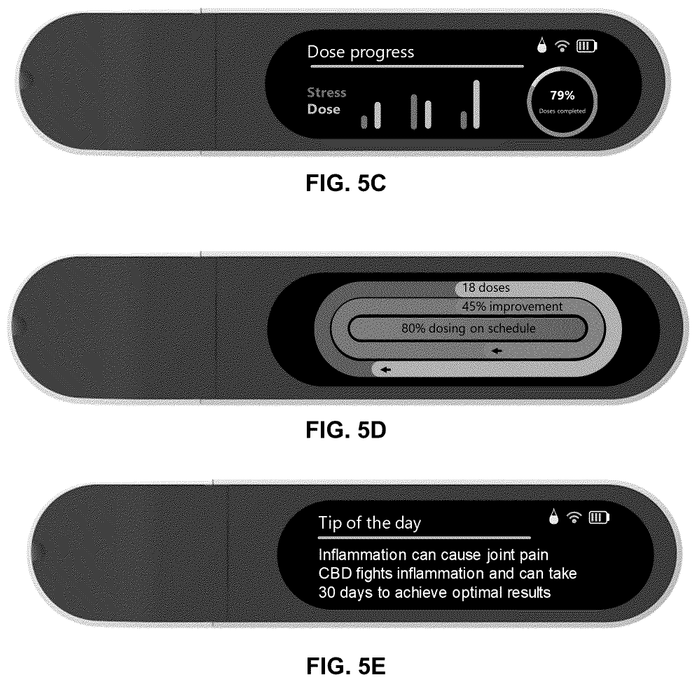

FIGS. 5A-5B show screen examples (on vaporizer device 100 and a user mobile device, respectively) of user history/progress, for example, dose and stress history. FIG. 5B illustrates how device 100 can interface with an application on a mobile device 502 that allows the user to further interact with information from, control, and communicate with device 100.

FIGS. 5C and B illustrate alternative displays of the progress illustrated in FIG. 5A. FIG. 5E illustrates that device 100 can be configured to provide tips or recommendations relative the reason the user is using device 100 and the selected media.

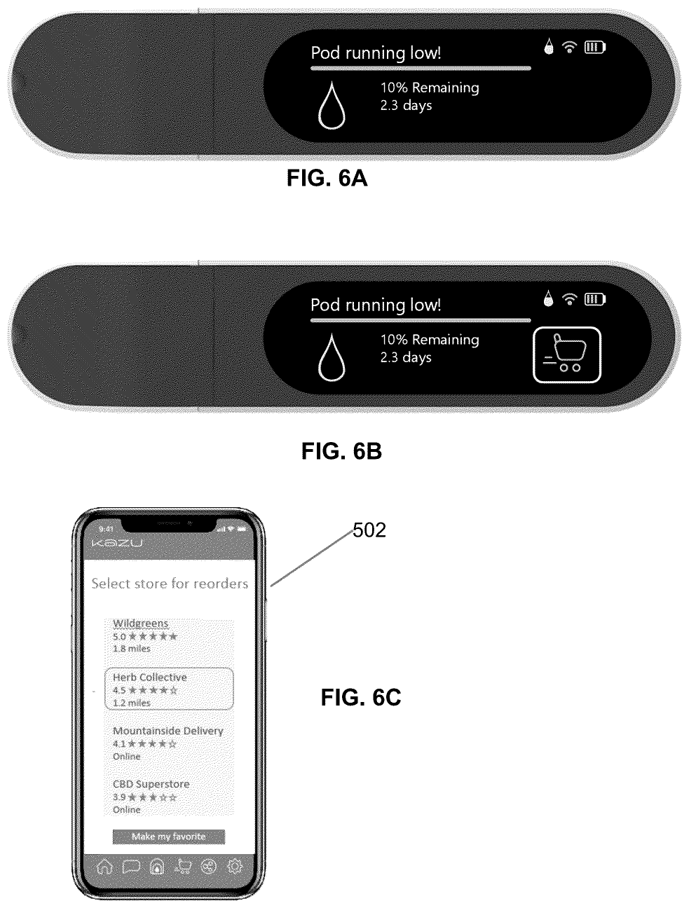

FIG. 6A shows a screen on the display 106 with a warning screen for the amount of inhalation media remaining in the cartridge/pod 104 running low. In an exemplary embodiment, the application 132 may monitor the amount of inhalation media in the pod and, if the amount dips below a threshold volume/percentage, causes the application 132 to show the warning screen. The warning screen may include a means to order a new pod (e.g., a shopping button shown in FIG. 6B). The application 132 may enable a user to pre-select/favorite stores or specific orders in the stores, for example, at setup. The pre-selected stores/orders may be linked to the ‘order’ button shown in FIG. 6B.

FIG. 6C illustrates that the application 132 can be interfaced with an application on a device 502 that allows the user to order and perform other functions to control, or in conjunction with device 100. Example functionality of the application running on device 502 are describe below. The application that runs on device 502 can also be considered an application 132, as both device 100 and device 02 can be considered a user system 130 in the architecture of FIG. 9.

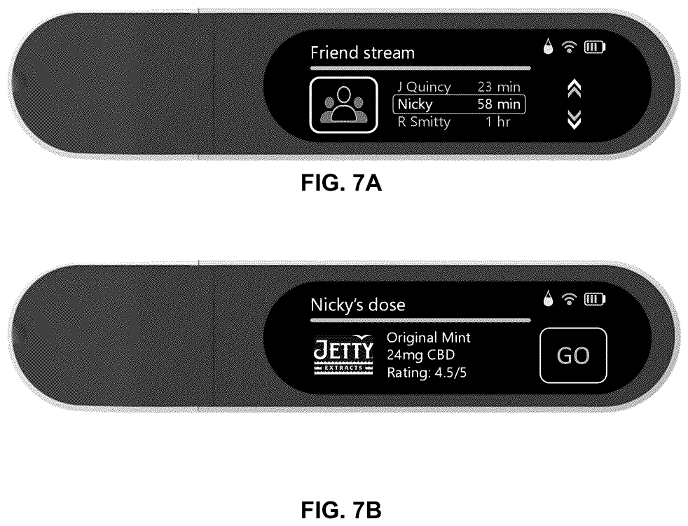

FIGS. 7A-7B show a list of social/friend channels (FIG. 7A) for a user and a specific dose from a specific friend (FIG. 7B). These screens may be accessed, for example, from a main menu (via icon 116 in FIGS. 1C-1D).

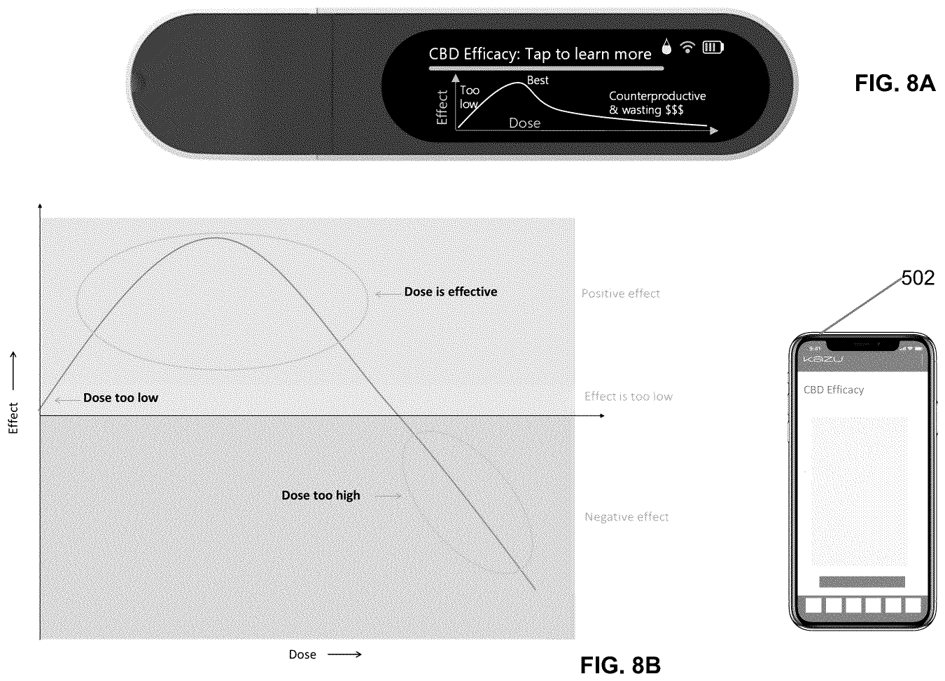

FIGS. 8A-8B show an analysis for a particular inhalation media that may be provided by the application 132 and which may be accessed by a user from the vaporizer device 100 or a user computer/mobile device. FIG. 8B illustrates how the same information can be accessed via the application on a device 502.

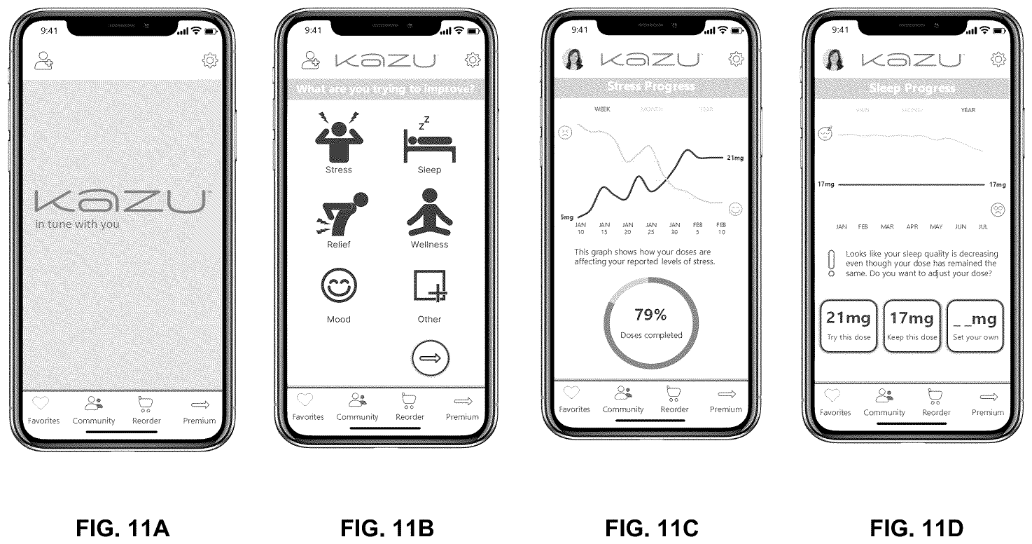

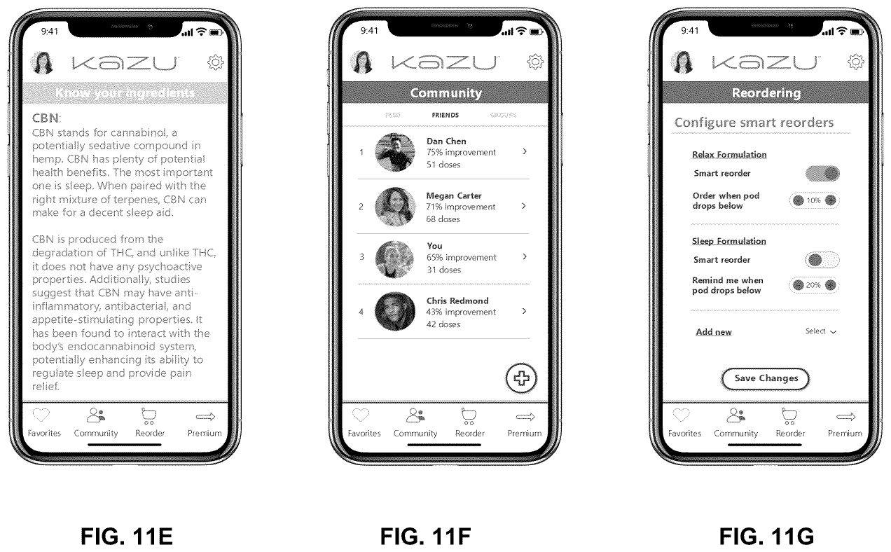

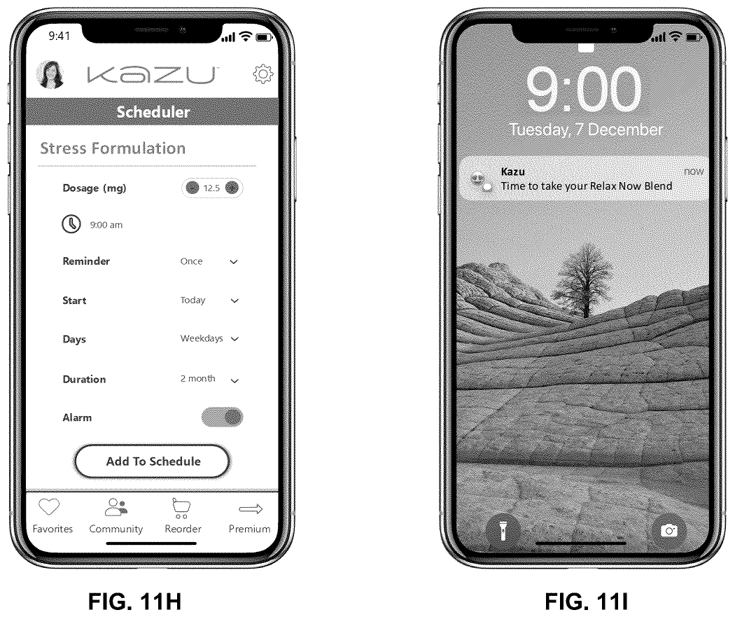

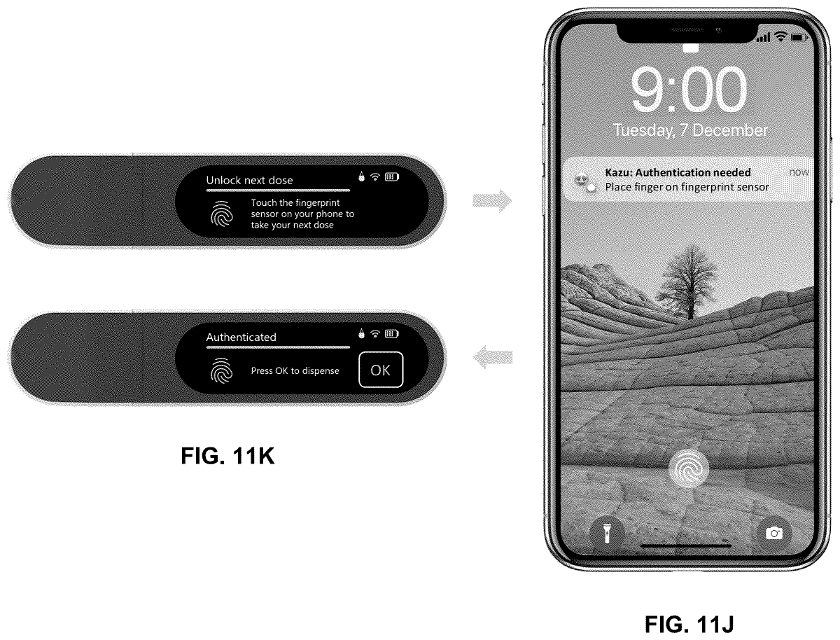

FIGS. 11A-k illustrate screen shots presented by an application (132) that can run a user system/device 130, as opposed to device 100, related to functionality provided by such an application. Some of this functionality overlaps with functionality that can be accessed via application 132 on device 100 and present via user interface 107. After accessing the opening screen as illustrated in FIG. 11A, it can be seen that the user can be presented with options to access their favorites, community, reorder capability, etc.

As illustrated in the screen shot of FIG. 11B, the user can select their purpose via the application, in addition to via device 100 illustrated in FIG. 2G. As illustrated in FIGS. 11C and D, progress can then be tracked and displayed to the user. As illustrated in FIG. 11E the user can be presented with ingredient information for the media being received via device 100. The user can curate, view, and interact with their community as illustrated in FIG. 11F. Recording can be set up as illustrate in FIG. 11G.

As illustrated in FIG. 11H, the user can set up a schedule for their dosing, which can result in reminders as illustrated in FIG. 11I.

FIGS. 11J and K illustrate an example of how the application on device 502 can work in conjunction with the device 100, in this case for access authentication.

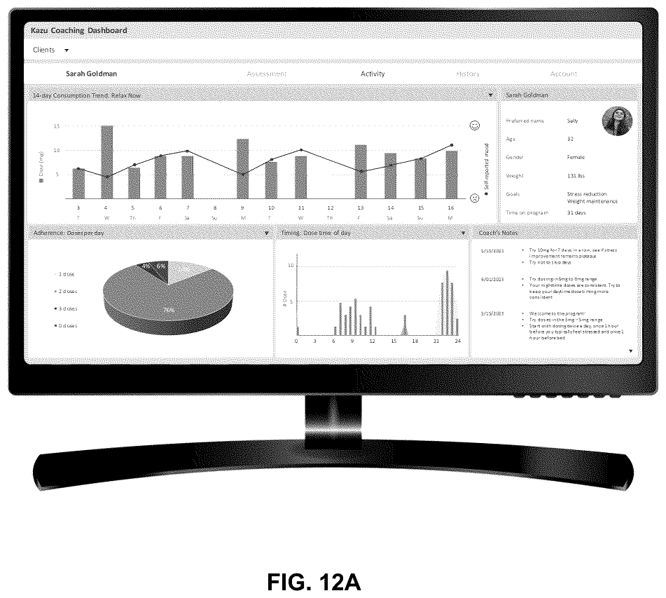

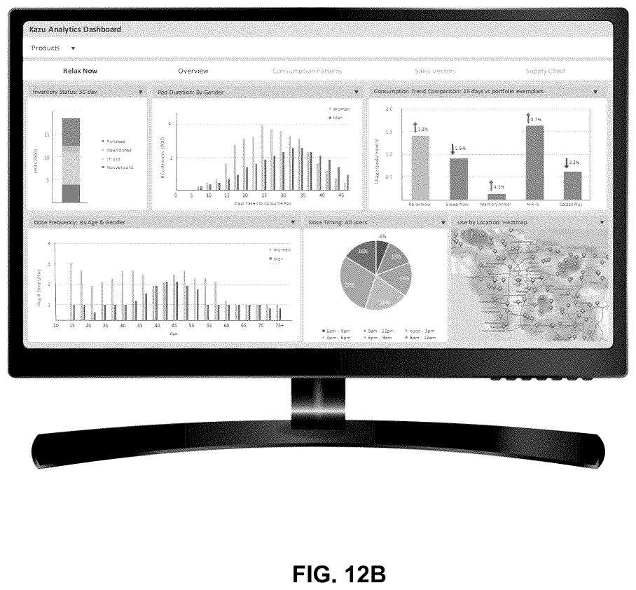

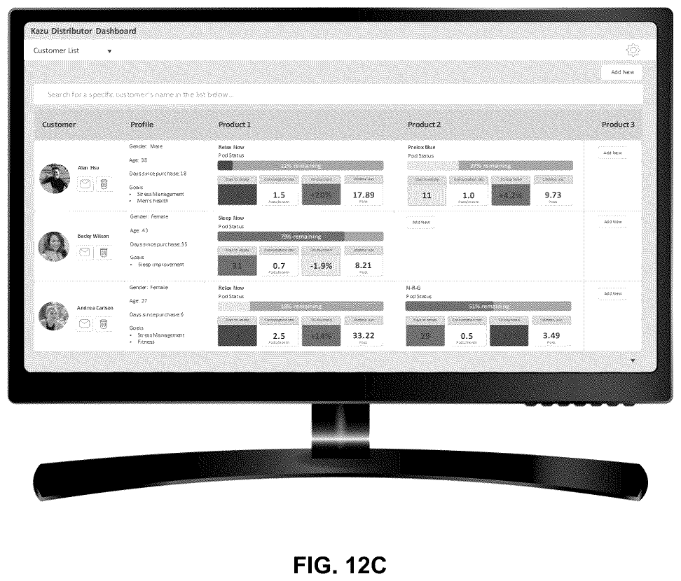

FIGS. 12A-C illustrate user interface screens that can be presented to, e.g., a coach (FIGS. 12A and B), to allow the coach to monitor and direct users, and a distributor (FIG. 12C) to allow them to ensure media availability for users.

Note that although the icons/options in various screens are presented in a specific order in the figures, the disclosure is not limited to any particular order or arrangement of icons representing the various options and/or information on the screen. Furthermore, the

FIG. 9 illustrates an example infrastructure in which one or more of the disclosed processes may be implemented, according to an embodiment. The infrastructure may comprise a platform 110, e.g., one or more servers, which hosts and/or executes one or more of the various functions, processes, methods, and/or software modules described herein. Platform 110 may comprise dedicated servers, or may instead comprise cloud instances, which utilize shared resources of one or more servers. These servers or cloud instances may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140, e.g., other platforms, websites, etc., via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

In an exemplary embodiment, user system(s) 130 include the media dispensing device 100 of the present disclosure. The user system(s) 130 may also comprise any type or types of computing devices capable of wired and/or wireless communication, including without limitation, desktop computers, laptop computers, tablet computers, smart phones or other mobile phones, and/or the like. Each user system 130 may comprise or be communicatively connected to the client application 132, discussed elsewhere in the disclosure, and/or one or more local databases 134. In an exemplary embodiment, the client application 132 includes a user interface which may be accessed via the display 106 of the vaporizer device 100. In one embodiment, the same or different client application 132 may be included on a user personal mobile device.

Much in the same way that the functionality and user experiences of a user mobile device or a vaporizer device 100 are defined by the applications running on said devices, the system described by the present invention can provide a variety of functionality and experiences that are determined by the applications running on the media dispensing device 100, a mobile device, and computer network 120 and the inhalation media.

Platform 110 may comprise web servers which host one or more websites and/or web services. In embodiments in which a website is provided, the website may comprise a graphical user interface, including, for example, one or more screens, e.g., webpages, generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols, e.g., HTTP, HTTPS, etc. These screens, e.g., webpages, may comprise a combination of content and elements, such as text, images, videos, animations, references, e.g., hyperlinks), frames, inputs, e.g., textboxes, text areas, checkboxes, radio buttons, drop-down menus, buttons, forms, etc., scripts, e.g., JavaScript, and the like, including elements comprising or derived from data stored in one or more databases, e.g., database(s) 114, that are locally and/or remotely accessible to platform 110. Platform 110 may also respond to other requests from user system(s) 130.

Platform 110 may comprise, be communicatively coupled with, or otherwise have access to one or more database(s) 114. For example, platform 110 may comprise one or more database servers which manage one or more databases 114. Server application 112 executing on platform 110 and/or client application 132 executing on user system 130 may submit data, e.g., user data, form data, etc., to be stored in database(s) 114, and/or request access to data stored in database(s) 114. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module, e.g., comprised in server application 112, executed by platform 110.

In embodiments in which a web service is provided, platform 110 may receive requests from external system(s) 140, and provide responses in eXtensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132, executing on one or more user system(s) 130, may interact with a server application 112 executing on platform 110 to execute one or more or a portion of one or more of the various functions, processes, methods, and/or software modules described herein. In an embodiment, client application 132 may utilize a local database 134 for storing data locally on user system 130.

Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. In an exemplary embodiment, processing of the client application 132 is carried out on the vaporizer device 100 (user system 130).

It should be understood that client application 132 may perform an amount of processing, relative to server application 112 on platform 110, at any point along this spectrum between “thin” and “thick,” depending on the design goals of the particular implementation. In any case, the software described herein, which may wholly reside on either platform 110, e.g., in which case server application 112 performs all processing, or user system(s) 130, e.g., in which case client application 132 performs all processing, or be distributed between platform 110 and user system(s) 130, e.g., in which case server application 112 and client application 132 both perform processing, can comprise one or more executable software modules comprising instructions that implement one or more of the processes, methods, or functions described herein.

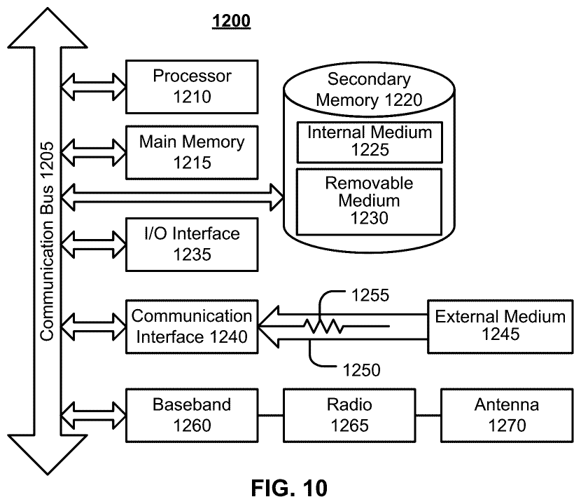

FIG. 10 is a block diagram illustrating an example wired or wireless system 1200 that may be used in connection with various embodiments described herein. For example, system 1200 may be used as or in conjunction with one or more of the functions, processes, or methods, e.g., to store and/or execute the software, described herein, and may represent components of platform 110, user system(s) 130, external system(s) 140, and/or other processing devices described herein. In an exemplary embodiment, the system 1200 is includes in the media dispensing device 100 discussed in the present application.

System 1200 can be a server or any conventional personal computer, or any other processor-enabled device that is capable of wired or wireless data communication. Other computer systems and/or architectures may be also used, as will be clear to those skilled in the art.

System 1200 preferably includes one or more processors 1210. Processor(s) 1210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms, e.g., digital-signal processor, a slave processor subordinate to the main processing system, e.g., back-end processor, an additional microprocessor or controller for dual or multiple processor systems, and/or a coprocessor.

Processor 1210 is preferably connected to a communication bus 1205. Communication bus 1205 may include a data channel for facilitating information transfer between storage and other peripheral components of system 1200. Furthermore, communication bus 1205 may provide a set of signals used for communication with processor 1210, including a data bus, address bus, and/or control bus (not shown). Communication bus 1205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 1200 preferably includes a main memory 1215 and may also include a secondary memory 1220. Main memory 1215 provides storage of instructions and data for programs executing on processor 1210, such as any of the software discussed herein. It should be understood that programs stored in the memory and executed by processor 1210 may be written and/or compiled according to any suitable language, including without limitation C/C++, Java, JavaScript, Perl, Visual Basic, .NET, and the like. Main memory 1215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 1220 is a non-transitory computer-readable medium having computer-executable code, e.g., any of the software disclosed herein, and/or other data stored thereon. The computer software or data stored on secondary memory 1220 is read into main memory 1215 for execution by processor 1210. Secondary memory 1220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 1220 may optionally include an internal medium 1225 and/or a removable medium 1230. Removable medium 1230 is read from and/or written to in any well-known manner. Removable storage medium 1230 may be, for example, a magnetic tape drive, a compact disc (CD) drive, a digital versatile disc (DVD) drive, other optical drive, a flash memory drive, and/or the like.

In alternative embodiments, secondary memory 1220 may include other similar means for allowing computer programs or other data or instructions to be loaded into system 1200. Such means may include, for example, a communication interface 1240, which allows software and data to be transferred from external storage medium 1245 to system 1200. Examples of external storage medium 1245 include an external hard disk drive, an external optical drive, an external magneto-optical drive, and/or the like.

As mentioned above, system 1200 may include a communication interface 1240. Communication interface 1240 allows software and data to be transferred between system 200 and external devices, e.g. printers, networks, or other information sources. For example, computer software or executable code may be transferred to system 1200 from a network server, e.g., platform 110, via communication interface 1240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 1200 with a network, e.g., network(s) 120, or another computing device. Communication interface 1240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 1240 are generally in the form of electrical communication signals 1255. These signals 1255 may be provided to communication interface 1240 via a communication channel 1250. In an embodiment, communication channel 1250 may be a wired or wireless network, e.g., network(s) 120, or any variety of other communication links. Communication channel 1250 carries signals 1255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code, e.g., computer programs, such as the disclosed software, is stored in main memory 1215 and/or secondary memory 1220. Computer-executable code can also be received via communication interface 1240 and stored in main memory 1215 and/or secondary memory 1220. Such computer programs, when executed, enable system 1200 to perform the various functions of the disclosed embodiments as described elsewhere herein.

In this description, the term “computer-readable medium” is used to refer to any non-transitory computer-readable storage media used to provide computer-executable code and/or other data to or within system 1200. Examples of such media include main memory 1215, secondary memory 1220 (including internal memory 1225 and/or removable medium 1230), external storage medium 1245, and any peripheral device communicatively coupled with communication interface 11240 (including a network information server or other network device). These non-transitory computer-readable media are means for providing software and/or other data to system 1200.

In an embodiment that is implemented using software, the software may be stored on a computer-readable medium and loaded into system 1200 by way of removable medium 1230, I/O interface 1235, or communication interface 1240. In such an embodiment, the software is loaded into system 1200 in the form of electrical communication signals 1255. The software, when executed by processor 1210, preferably causes processor 1210 to perform one or more of the processes and functions described elsewhere herein.

In an embodiment, I/O interface 1235 provides an interface between one or more components of system 1200 and one or more input and/or output devices. In an exemplary embodiment, the I/O interface 1235 comprises the display screen 106 of the media dispensing device 100 (FIG. 1A).

Other example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display, e.g., in a smartphone, tablet, or other mobile device.

System 1200 may also include optional wireless communication components that facilitate wireless communication over a voice network and/or a data network, e.g., in the case of user system 130. The wireless communication components comprise an antenna system 1270, a radio system 1265, and a baseband system 1260. In system 1200, radio frequency (RF) signals are transmitted and received over the air by antenna system 1270 under the management of radio system 1265.

In an embodiment, antenna system 1270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 1265.

In an alternative embodiment, radio system 1265 may comprise one or more radios that are configured to communicate over various frequencies. In an embodiment, radio system 1265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 1265 to baseband system 1260.

While the disclosure has been described in terms of exemplary embodiments, those skilled in the art will recognize that the disclosure can be practiced with modifications in the spirit and scope of the appended claims. These examples given above are merely illustrative and are not meant to be an exhaustive list of all possible designs, embodiments, applications, or modifications of the disclosure.

What is claimed is:

1. A system for dispensing media, comprising:
a device comprising:
a driver component having an end configured to act on a movable surface of a cartridge,
a motor configured to controllably position the driver component,
a display,
at least one hardware processor, and
one or more software modules that are configured to, when executed by the at least one hardware processor, receive media dose information and control the motor based on the media dose information in order to dispense a dose of the media;
a cartridge having:
a media storage area for storing the media,
at least one movable surface that changes the volume of the media stored in the media storage area under control of the driver component, and
an outlet through which the media is dispensed in order to change the volume of the media stored in the media storage area under control of the driver component.

2. The system of claim 1, wherein the display screen used to control the amount of media that is dispensed.

3. The system of claim 1, wherein the display screen displays information about the contents of the cartridge.

4. The system of claim 1, wherein the cartridge further comprises programmable memory.

5. The system of claim 4, wherein the programmable memory is configured to store a value associated with the amount of media contained in the media storage area.

6. The system of claim 5, wherein the display screen displays information associated with a value stored within the programmable memory of the cartridge.

7. The system of claim 1, wherein the display screen displays consumption history information.

8. The system of claim 1, wherein the display screen has the ability to detect touch input.

9. The system of claim 8, wherein the touch input of the display screen is used to receive input from the user.

10. The system of claim 1, wherein the display screen is configured to display a list of multiple doses from which to select.

11. The system of claim 1, wherein the display screen is configured to determine how the user feels.

12. The system of claim 1, wherein the display screen is configured to suggest a dose to the user.

13. The system of claim 1, wherein the display screen is configured to collect feedback from the user regarding the dose.

14. The system of claim 13, wherein the display screen is configured to allow the user to share dosing information via a network.

15. The system of claim 13, wherein the display screen is configured to allow the user to schedule the timing of doses.

16. The system of claim 1, wherein the display screen is configured to display the progress toward a target.

17. The system of claim 1, wherein the display screen is configured to display advice regarding the consumption of the media.

18. The system of claim 1, wherein the display screen is configured to allow the user to purchase cartridges.

19. The system of claim 1, wherein the display screen is configured to allow the user to select other users within the network for the purposes of sharing dose information.

20. The system of claim 1, wherein the display screen is configured to allow the user to mark certain doses for reuse in the future.

21. The system of claim 1, where the display screen is configured to display consumption information from other users within the network.

22. The system of claim 1, wherein the media dose information displayed on the display screen is based, at least in part, on information read from the programmable memory.

23. The system of claim 1, wherein the media dose information displayed on the display screen is based, at least in part, on background information related to a user.

24. The system of claim 1, wherein the media dose information displayed on the display screen is based, at least in part, on a selection by the user.

25. The system of claim 1, wherein the media dose information displayed on the display screen is based, at least in part, on information received from or provided by a third party.

26. The system of claim 1, wherein the media dose information displayed on the display screen is based, at least in part, by determination of a data analysis program.

27. The system of claim 1, wherein the at least one constituent comprises at least one of Melatonin, CBD, CBN, Nicotine or THC.

28. The system of claim 1, wherein the media is an ingested media.

29. The system of claim 1, wherein the cartridge has the ability to vaporize the media to be inhaled by the user.
